(19) United States
(12) Patent Application Publication
(10) Pub. No.: US 2026/0202403 A1
(43) Pub. Date: Jul. 16, 2026

(54) METHODS OF DIAGNOSING AND TREATING NEUROPSYCHIATRIC DISEASES AND DISORDERS

(71) Applicant: Yale University, New Haven, CT (US)

(72) Inventors: Christopher Pittenger, New Haven, CT (US); Jian Xu, New Haven, CT (US); Aaron Ring, New Haven, CT (US)

(21) Appl. No.: 19/134,653

(22) PCT Filed: Nov. 30, 2023
(86) PCT No.: PCT/US2023/081787

§ 371 Date: May 30, 2025

Related U.S. Application Data
(60) Provisional application No. 63/429,467, filed on Dec. 1, 2022.

(51) Int. Cl. G01N 33/564; A61K 45/06; C07K 16/28

(52) U.S. Cl. CPC G01N 33/564; A61K 45/06; C07K 16/2887; G01N 2333/49; G01N 2333/521; G01N 2333/705; G01N 2800/30

(57) ABSTRACT

Provided herein are methods of diagnosing neuropsychiatric diseases and disorders by means of identifying antigens that bind to specific cells in whole neural tissue. In a non-limiting example, a method of diagnosing pediatric acute-onset neuropsychiatric syndrome (PANS) is provided.

### CROSS-REFERENCE TO RELATED APPLICATION

This application claims priority to U.S. Provisional Patent Application Ser. No. 63/429,467 entitled “METHODS OF DIAGNOSING AND TREATING NEUROPSYCHIATRIC DISEASES AND DISORDERS,” filed Dec. 1, 2022, the disclosure of which is incorporated herein by reference in its entirety.

### STATEMENT REGARDING FEDERALLY SPONSORED RESEARCH

This invention was made with government support under contract nos. NS101104, MH109700, MH118453, NS114166, and NS133434 awarded by National Institutes of Health. The government has certain rights in the invention.

### BACKGROUND

In some instances, children develop symptoms of OCD (obsessive-compulsive disorder) and tics very rapidly, even overnight. This type of neuropsychiatric disorder is known as Pediatric Acute-onset Neuropsychiatric Syndrome, or PANS. It has long been hypothesized that this can result from an autoimmune process triggered by an infection. When the infection is Streptococcus (in which context this phenomenon was first characterized), it is known as Pediatric Autoimmune Disorder Associated with Streptococcus, or PANDAS. These disorders are frequently diagnosed in children, but the clinical landscape is uncertain, and the research base has poor evidentiary support.

The field is in need of a better biological understanding of these phenomena, as well as diagnostic tests that can help define more homogeneous patient populations, so that children suffering from these disorders can be adequately treated. Clinically, and commercially, a well validated test that can help clarify diagnosis would be of significant utility.

The present disclosure addresses this unmet need.

### BRIEF SUMMARY

In various aspects, a method for diagnosing a neuropsychiatric disease or disorder in a human subject is provided. In certain embodiments, the method comprises obtaining a serum sample comprising IgG from the human subject. In certain embodiments, the method comprises quantifying using ELISA the amount of an autoantibody binding to the target antigen set, so as to provide an ELISA value. In certain embodiments, the autoantibody comprises at least one of an anti-LRP11 antibody, an anti-CXCL3 antibody, an anti-PDGFB antibody, and an anti-CSPG5 antibody. In certain embodiments, the method comprises diagnosing the human subject with PANS if the ELISA value is greater than or equal to 3 standard deviations above a control mean ELISA value.

In various aspects, a method for identifying a disease-inducing agent in a neuropsychiatric disease or disorder is provided. In certain embodiments, the method comprises providing an isolated tissue sample containing cholinergic interneurons (CINs). In certain embodiments, the method comprises identifying one or more antibodies in a serum specimen that bind to the CINs. In certain embodiments, the method comprises quantifying using ELISA the amount of the one or more antibodies that bind to the CINs so as to provide an ELISA value. In certain embodiments, the method comprises comparing the ELISA value against a control ELISA value to determine whether the one or more antibodies are disease-inducing agents.

### BRIEF DESCRIPTION OF THE FIGURES

The drawings illustrate generally, by way of example, but not by way of limitation, various embodiments of the present application.

FIGS. 1A-1C show elevated binding to CINs by IgG from children with PANDAS. FIG. 1A: Immunofluorescence was used to quantify colocalization of IgG binding from PANDAS and control serum (green) with ChAT (red) in mouse striatal slices. FIG. 1B: Elevated IgG binding to CINs was seen in slices treated with PANDAS serum relative to matched controls, across three cohorts of subjects (t[48]=2.67, p=0.01). FIG. 1C: No such elevated binding was seen to other tested cell types, including cholinergic neurons in the medial septal nucleus, shown here. Data from Xu, J. et al. Antibodies From Children With PANDAS Bind Specifically to Striatal Cholinergic Interneurons and Alter Their Activity. The American journal of psychiatry 178, 48-64, doi:10.1176/appi.ajp.2020.19070698 (2021). Different colors correspond to three cohorts of patients, assayed separately and normalized to control mean.

FIG. 2A shows representative images of immunohistochemical staining of human IgG (green) and parvalbumin (PV, red). Arrowheads indicate human IgG binding to PV-positive interneurons.

FIG. 2B shows plasma IgG binding to PV interneurons did not significantly differ between PANS “flare” and control groups (2-tailed independent sample t-test: t[48]=1.915, p=0.061).

FIG. 2C shows representative confocal images of immunohistochemical staining of human IgG (green) and parvalbumin (PV, red) in human caudate.

FIG. 2D illustrates that PANS “flare” IgG did not show elevated binding to PV-positive interneurons. In caudate, a significant main effect was found (one-way ANOVA: F(3, 20)=11.42, p=0.0001, η²=0.631). IgG binding in the PANS “flare” group did not differ from that of the control group (Control vs Flare, p=0.987) or the “recovered” group (Flare vs Recovered, p=0.445). No difference was found between the control and “recovered” groups (Control vs Recovered, p=0.278). All three group showed higher IgG fluorescence intensity relative to PANS “flare” plasmas from which IgG was depleted (Control vs IgG-dep, p=0.0002; Flare vs IgG-dep, p=0.0005; Recovered vs IgG-dep, p=0.015).

FIGS. 3A-3C show reduced IgG binding to CINs after symptom improvement in PANDAS. FIG. 3A: IgG binding to CINs was reduced after treatment in 100% of 11 PANDAS subjects who responded to IVIG (t[11]=8.19, p<0.0001). FIG. 3B: Symptom improvement (CY-BOCS) correlated tightly with change in IgG binding to CINs (r²=0.762, p=0.0005). Data from Xu, J. et al. Antibodies From Children With PANDAS Bind Specifically to Striatal Cholinergic Interneurons and Alter Their Activity. Amer. J. Psych. 178, 48-64, doi:10.1176/appi.ajp.2020.19070698 (2021). FIG. 3C: In the Stanford cohort, CIN binding by IgG in serum collected during flare and during later remission was similarly reduced (t[12]=4.46, p=0.005).

FIG. 4 shows CIN activity, indexed by P-rpS6, is reduced by pretreatment with PANDAS baseline serum (S1). Post-IVIG serum from the same subjects (S3) and baseline serum depleted of IgG (S1-dep) did not have this effect. ***p<0.001; *p<0.0001. Data from Xu, J. et al. Antibodies From Children With PANDAS Bind Specifically to Striatal Cholinergic Interneurons and Alter Their Activity. Amer. J. Psych. 178, 48-64, doi:10.1176/appi.ajp.2020.19070698 (2021).

FIG. 5 shows a schematic overview of the REAP (Rapid Exoproteome Antibody Profiling) workflow.

FIGS. 6A-6B show elevated anti-LRP11 in PANDAS samples by ELISA. FIG. 6A: Anti-LRP11 was elevated in 50 PANDAS samples from NIMH compared to 23 controls (p<0.0001), including in the 27 subjects described in FIGS. 1A-1C. Dotted line indicates 3 SD above the mean of the control group; 27 of 50 PANDAS samples (0 controls; 14 of the 27 in FIGS. 1A-1C) fell above this cutoff. FIG. 6B: Anti-LRP11 antibodies were similarly elevated in PANDAS cases from the Stanford sample (p=0.004. Here the 3SD cutoff was higher due to variance in the control group; nevertheless 3 of 25 PANDAS samples had anti-LRP11 antibody levels above this cutoff).

FIGS. 7A-7E show Anti-LRP11 antibody in PANDAS. FIG. 7A: Anti-LRP11 IgG purified from a selected PANDAS serum (left) and commercial anti-LRP11 antibody (right) both bind to CINs, as well as other striatal cells, in mouse tissue. FIG. 7B: Both antibodies similarly bind to CINs in human post-mortem striatum. FIG. 7C: Both anti-LRP11 IgG purified from PANDAS sera and commercial anti-LRP11 bind more to CINs than to PV-interneurons (2-way ANOVA: main effect of neuron type: p<0.0001; main effect of Ab and interaction NS. FIG. 7D: IgG binding to CINs (multiple cells from a single serum, visualized at 10X) was reduced by depletion of either all IgG or only anti-LRP 11 Ab, and persisted when slices were treated with purified IgG or, to a lesser extent, with purified anti-LRP11 Ab. 1-way ANOVA: F[7,98]=17.4, p<0.0001. FIG. 7E shows that anti-LRP11 antibodies contribute to the CIN binding in multiple anti-LRP11-positive samples. 1-way ANOVA: F[3,36]=25.59, p<0.0001.

FIG. 8 shows a non-limiting embodiment of a strategy for identification and cloning of antigen-specific autoantibodies.

FIGS. 9A-9C shows purification of patient-specific autoantibodies in myasthenia gravis. These data are shown to establish feasibility of the approach to isolating B-cell clones, and thus specific autoantibodies, that we propose here. Plots show flow cytometric analysis of the cell-based assay for antibody binding. HEK cells were transfected with AChR. The x-axis shows GFP fluorescence, which reflects transfection with AChR. The y-axis shows anti-human IgG Fc antibody binding, which reflets primary binding to AChR on transfected HEK cells. FIG. 9A. Positive control anti-AChR mAb 637. FIG. 9B. Culture medium from a B cell from a control subject. FIG. 9C. Culture medium from a B-cell clone producing anti-AChR mAb from a patient with myasthenia gravis.

FIGS. 10A-10B show binding of antibodies to PDGFB (platelet-derived growth factor subunit B) (FIG. 10A) and CXCL3 (CXC motif chemokine ligand 3) (FIG. 10B) and elevated binding to these targets in two sets of PANS/PANDAS samples.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 w t% or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 w t% or less. The term “substantially free of” can mean having a trivial amount of, such that a composition is about 0 wt% to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “patient,” “subject,” or “individual” are used interchangeably herein, and refer to any animal, or cells thereof whether in vitro or in situ, amenable to the methods described herein. In a non-limiting embodiment, the patient, subject, or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve, or affect a condition contemplated herein, or the symptoms of a condition contemplated herein. Such treatments may be specifically tailored or modified, based on knowledge obtained from the field of pharmacogenomics.

### Methods of Diagnosing, Testing, Treating, Ameliorating, And/or Preventing Neuropsychiatric Diseases and Disorders

In various embodiments, a method for diagnosing a neuropsychiatric disease or disorder is provided. In various embodiments, the neuropsychiatric disease or disorder is pediatric acute-onset neuropsychiatric syndrome (PANS) and/or pediatric autoimmune disorder associated with Streptococcus (PANDAS).

In certain embodiments, the diagnostic tests described herein target at least one of LRP11, CXCL3, and/or CSPG5 antigens. In other embodiments, the autoantibody comprises at least one of anti-LRP11 antibody, anti-CXCL3 antibody, anti-PDGFB antibody, and/or anti-CSPG5 antibody. In other embodiments, the autoantibody comprises an anti-LRP11 antibody. In other embodiments, the autoantibody comprises an anti-CXCL3 antibody. In other embodiments, the autoantibody comprises an anti-CSPG5 antibody. In other embodiments, the autoantibody comprises an anti-PDGFB antibody. In certain embodiments, the method comprises diagnosing the human subject with a neuropsychiatric disease or disorder, such as PANS, if the ELISA value is greater than or equal to 3 standard deviations above a control mean ELISA value.

In various embodiments, the target antigen is LRP11. In various embodiments, the autoantibody is an anti-LRP11 antibody. In various embodiments, the target antigen is CXCL3. In various embodiments, the autoantibody is an anti-CXCL3 antibody. In various embodiments, the target antigen is CSPG5. In various embodiments, the autoantibody is an anti-CSPG5 antibody. In various embodiments, the target antigen is PDGFB. In various embodiments, the autoantibody is an anti-PDGFB antibody.

In various embodiments, the method includes using the antibody test described herein to guide the treatment of the human subject to reduce or ameliorate at least one symptom of PANS. In various embodiments, treating includes administering to the human subject a therapeutically effective amount of IVIG (intravenous immunoglobulin). Contemplated herein are treatment(s) that include other immunomodulatory agents that can reduce or ameliorate at least one symptom of PANS. Other suitable treatments can include, but are not limited to, plasmapheresis, rituximab, steroids, and non-steroidal anti-inflammatory drugs (NSAIDs).

In various embodiments, the human subject is a child that is 6 months to 17 years of age. In various embodiments, the child is at least about, greater than, or about equal to 6 months, 1, 2, 3, 4, 5, 6, 7, 8, 9, 10, 11, 12, 13, 14, 15, 16, or 17 years of age. In various embodiments, the child is an age bracket formed by any of the foregoing ages. In various embodiments, the human subject is an adult aged 18 or older. In various embodiments, the human subject is a child aged 3-12 years.

The methods described herein include administering to the subject a therapeutically effective amount of at least one compound described herein, such as IVIG, rituximab, steroids, or NSAIDs, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats, ameliorates, and/prevents a neuropsychiatric disease or disorder as described herein.

Suitable steroids include, but are not limited to, hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, acleometasone dipropionate, betamethasone valerate, betamethasone dippropionate, prednicarbate, clobetasone-17-butyrate, clobetasol-17-propionate, fluocortilone caproate, fluocortolone pivalate, and fluprednidene acetate, hydrocortisone- 17-butyrate, 17-aceponate, 17-buteprate, and prednicarbate.

Steroids can be administered in any dose described herein, for example, the dose of a steroid can be about 1-500 mg, 5 -25 mg, about 1-3 mg, about 2-4 mg, about 3-5 mg, about 4-6 mg, about 5-7 mg, about 6-8 mg, about 7-9 mg, about 8-10 mg, about 10-15 mg, about 10-20 mg, about 20-50 mg, about 50-100 mg, about 100-200 mg, about 200-300 mg, about 300-400 mg, 400-500 mg 1-20 mg, about 10-30 mg, about 20-40 mg, about 30-50 mg, about 40-60 mg, about 50-70 mg, about 60-80 mg, about 70-90 mg, about 80-100 mg, about 90-110 mg, about 100-120 mg, about 110-130 mg, about 120-140 mg, about 130-150 mg, about 140-160 mg, about 150-170 mg, about 160-180 mg, about 170-190 mg, about 180-200 mg, about 190-210 mg, about 200-220 mg, about 210-230 mg, about 220-240 mg, about 230-250 mg, about 240-260 mg, about 250-270 mg, about 260-280 mg, about 270-290 mg, about 280-300 mg, about 290-310 mg, about 300-320 mg, about 310-330 mg, about 320-340 mg, about 330-350 mg, about 340-360 mg, about 350-370 mg, about 360-380 mg, about 370-390 mg, about 380-300 mg, about 390-410 mg, about 400-420 mg, about 410-430 mg, about 420-440 mg, about 430-450 mg, about 440-460 mg, about 450-470 mg, about 460-480 mg, about 470-490 mg, about 480-300 mg, about 490-510 mg of the steroid, or any amount in a range bounded by any of these values. Steroids can be dosed any suitable route of administration described herein, for example oral administration or intravenous administration through an infusion.

Steroids can be dosed multiple times per day, such as for example one to four times per day. Suitable NSAIDs include, but are not limited to, Propionic acid drugs such as Fenoprofen calcium (Nalfon®), Flurbiprofen (Ansaid®), Suprofen. Benoxaprofen, Ibuprofen (prescription Motrin®), Ibuprofen (200 mg. over the counter Nuprin, Motrin 1B®), Ketoprofen (Orduis, Oruvall®), Naproxen (Naprosyn®), Naproxen sodium (Aleve, Anaprox, Aflaxen®), Oxaprozin (Daypro®), or the like; Acetic acid drug such as Diclofenac sodium (Voltaren®), Diclofenac potassium (Cataflam®), Etodolac (Lodine®), Indomethacin (Indocin®), Ketorolac tromethamine (Acular, Toradol® intramuscular), Ketorolac (oral Toradol®), or the like; Ketone drugs such as Nabumetone (Relafen®), Sulindac (Clinoril®), Tolmetin sodium (Tolectin®). or the like; Fenamate drugs such as Meclofenamate sodium (Meclomen®), Mefenamic acid (Ponstel®), or the like; Oxicam drugs such as Piroxicam (Dolibid®), or the like; Salicylic acid drugs such as Diflunisal (Feldene®), Aspirin, or the like; Pyrazolin acid drugs such as Oxyphenbutazone (Tandearil®), Phenylbutazone (Butazolidin®), or the like; acetaminophen (Tylenol®), or the like; COX-2 inhibitors such as Celebrex, Vioxx, or the like, or mixtures or combinations thereof.

NSAIDs can be administered in any dose described herein, for example, the dose of a NSAID can be about 1-500 mg, 5 -25 mg, about 1-3 mg, about 2-4 mg, about 3-5 mg, about 4-6 mg, about 5-7 mg, about 6-8 mg, about 7-9 mg, about 8-10 mg, about 10-15 mg, about 10-20 mg, about 20-50 mg, about 50-100 mg, about 100-200 mg, about 200-300 mg, about 300-400 mg, 400-500 mg 1-20 mg, about 10-30 mg, about 20-40 mg, about 30-50 mg, about 40-60 mg, about 50-70 mg, about 60-80 mg, about 70-90 mg, about 80-100 mg, about 90-110 mg, about 100-120 mg, about 110-130 mg, about 120-140 mg, about 130-150 mg, about 140-160 mg, about 150-170 mg, about 160-180 mg, about 170-190 mg, about 180-200 mg, about 190-210 mg, about 200-220 mg, about 210-230 mg, about 220-240 mg, about 230-250 mg, about 240-260 mg, about 250-270 mg, about 260-280 mg, about 270-290 mg, about 280-300 mg, about 290-310 mg, about 300-320 mg, about 310-330 mg, about 320-340 mg, about 330-350 mg, about 340-360 mg, about 350-370 mg, about 360-380 mg, about 370-390 mg, about 380-300 mg, about 390-410 mg, about 400-420 mg, about 410-430 mg, about 420-440 mg, about 430-450 mg, about 440-460 mg, about 450-470 mg, about 460-480 mg, about 470-490 mg, about 480-300 mg, about 490-510 mg of the NSAID, or any amount in a range bounded by any of these values. NSAIDs can be dosed any suitable route of administration described herein, for example oral administration or intravenous administration through an infusion. NSAID scan be dosed multiple times per day, such as for example one to six times per day.

In certain embodiments, the compound(s) described herein and the therapeutic agent are co-administered to the subject. In other embodiments, the compound(s) described herein and the therapeutic agent are coformulated and co-administered to the subject.

In various embodiments, a method for identifying a disease-inducing agent in a neuropsychiatric disease or disorder is provided.

In certain embodiments, the method comprises providing an isolated tissue sample containing cholinergic interneurons (CINs). In certain embodiments, the method comprises identifying one or more antibodies in a serum specimen that bind to the CINs. In certain embodiments, the method comprises quantifying using ELISA, or another technique capable of quantifying antibodies, the amount of one or more antibodies that bind to the CINs to provide an ELISA value (quantified value). In certain embodiments, the method comprises comparing the ELISA value (quantified value) against a control ELISA value (control value) to determine whether the one or more antibodies are disease-inducing agents.

In various embodiments, the one or more antibodies includes an LRP11 antibody.

In various embodiments, the one or more antibodies includes an CXCL3 antibody.

In various embodiments, the one or more antibodies includes an CSPG5 antibody.

In various embodiments, the one or more antibodies includes an PDGFB antibody.

In various embodiments, the isolated tissue sample is human tissue.

In various embodiments, the neuropsychiatric disease or disorder is PANS.

In various embodiments, the neuropsychiatric disease or disorder is PANDAS.

In certain embodiments, the subject is a mammal. In other embodiments, the mammal is a human.

Obsessive-compulsive disorder (OCD) affects approximately one out of 40 people and causes enormous morbidity; it is a leading cause of disability in the developed world. Prevalence has been estimated as 1-4% in the pediatric population. Pediatric OCD can badly disrupt normal psychological and social development. Available treatments-both psychotherapeutic and pharmacological-are of benefit to many children and adults, but a substantial minority are refractory even to optimal treatment.

A subset of cases of pediatric OCD have a strikingly acute onset and can even manifest overnight; these patients often have a fluctuating clinical course. This presentation has been dubbed ‘pediatric acute-onset neuropsychiatric syndrome’, or PANS. The striking natural history of PANS patients may suggest a unique pathophysiology. A temporal association with infectious illness in many cases has suggested the possibility of a neuroinflammatory etiology; association specifically with a Streptococcal infection underlies the older diagnosis ‘pediatric autoimmune neuropsychiatric disorder associated with Streptococcus’ (PANDAS). It has been suggested infection can, in a susceptible host, lead to the production of antibodies that cross-react with brain antigens, thereby producing neuroinflammation and neuropsychiatric symptomatology. An analogy is often made to Sydenham's chorea, in which antibody-mediated pathophysiology after Streptococcal infection is broadly accepted. There may also be an analogy to more recent work on COVID, in which infection-triggered production of autoantibodies has recently been described; some of these cross-react with brain antigens and produce neuropsychiatric symptomatology. However, this pathophysiological hypothesis for PANS and PANDAS remains unproven, and controversial in some circles.

Importantly, neuroinflammatory pathophysiology may not be restricted to PANS but may occur in a broader subset of OCD cases. Anti-basal ganglia antibodies have been reported in OCD cohorts more generally, and a recent PET imaging study suggests the presence of activated microglia in the basal ganglia circuitry implicated in adult OCD. A focus on PANS may limit phenotypic heterogeneity and thus facilitate the identification of specific pathophysiological mechanisms, but insights derived from the study of PANS and PANDAS may generalize to adult patients, and to other diagnoses.

### Identifying Additional Antibodies

New antibodies can be identified by obtaining a serum sample comprising IgG from the human subject. The serum sample can be screened using a yeast surface display library that includes at least one target antigen expressed in the brain or other organ of interest. The screening also includes quantifying using ELISA the amount of an autoantibody binding to the target antigen set to provide an ELISA value.

The yeast surface display library includes providing a genetically barcoded library of extracellular or secreted proteins displayed on yeast cells. The step of using the yeast surface display library includes contacting the serum sample with an array of microtiter plates having the yeast cells that include the genetically barcoded library. The step of using the yeast surface display library also includes isolating IgG bound to the library proteins using high throughput magnetic selection to identify the bound antigens. The step of using the yeast surface display library also includes obtaining a quantitative readout of the bound antigens by sequencing the genetically barcoded library.

### Anti-basal Ganglia Interneuron Antibodies in Pans

Antibody binding was examined in intact brain tissue (using mice as a convenient model system but then replicating in human brain tissue), rather than in reduced systems.

The focus of the antibody binding was on which cells serum antibodies bind to, rather than which molecular targets. Without being bound by theory, it is believed that binding to distinct epitopes or molecular targets on the same cells might have similar pathogenic effects on circuit function.

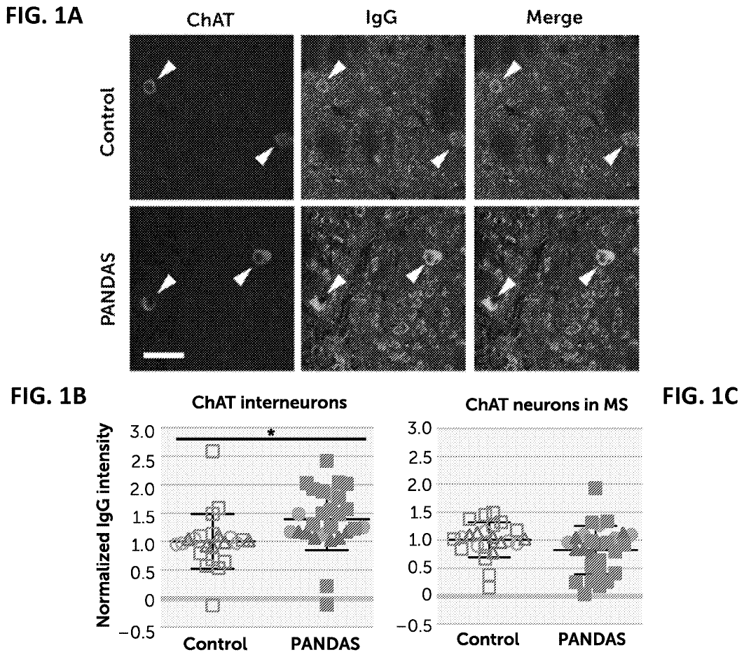

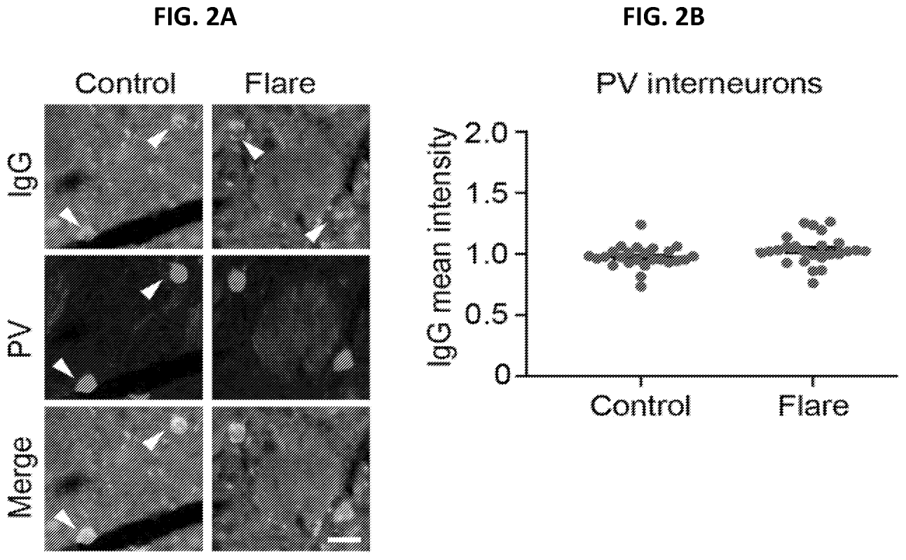

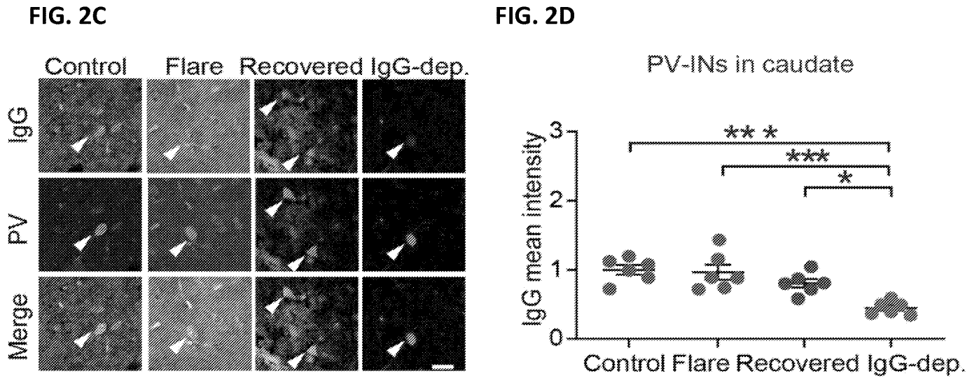

Antibodies in sera from patients, diluted to IgG concentration of 1.25 mg/dL, exhibited elevated binding to a specific subtype of interneuron in the mouse striatum, the cholinergic interneurons (CINs), as show in FIGS. 1A-1C. These results were further replicated in human post-mortem striatal tissue. Binding to a number of other neuron types was also examined, including D1R-and D2R-expressing spiny projection neurons in the striatum, parvalbumin-and N-NOS-expressing GABAergic interneurons in the striatum, and cholinergic projection neurons in the adjacent medial septal nuclei; there was no elevation of binding by PANDAS IgG to any of these interneuron types, demonstrating that elevated binding to CINs is notably specific. This level of rigor and replication are particularly important given the history of non-replication in this field in the past. This elevated binding was replicated in another cohort of patients, from a different clinic (the ‘Stanford cohort’; FIG. 2).

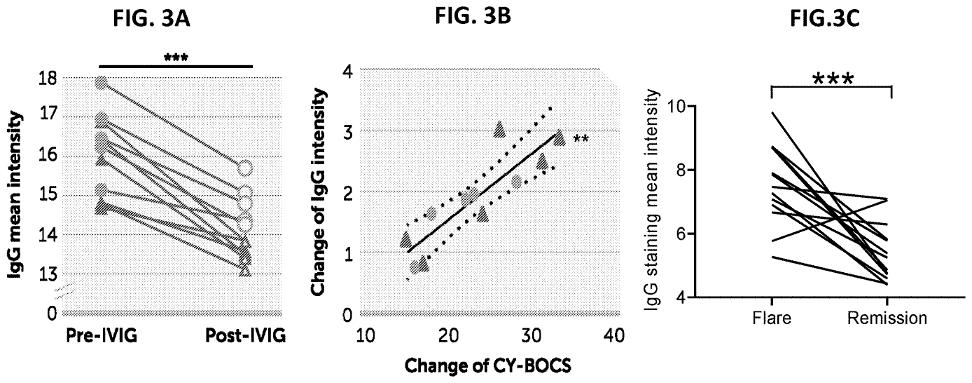

The relationship of this binding to symptomatology was examined by quantifying binding at baseline and after immunomodulatory treatment with IVIG. IgG binding to CINs was reduced after IVIG, and this reduction correlated tightly with symptom improvement (FIGS. 3A-3B). No change was seen in binding to parvalbumin-expressing GABAergic interneurons. In the Stanford cohort, patients were treated clinically using a variety of interventions; CIN binding in the same children after symptom remission was similarly reduced (FIG. 3C), with no change in binding to PV interneurons.

### Pandas-Associated Antibodies Reduce CIN Activity

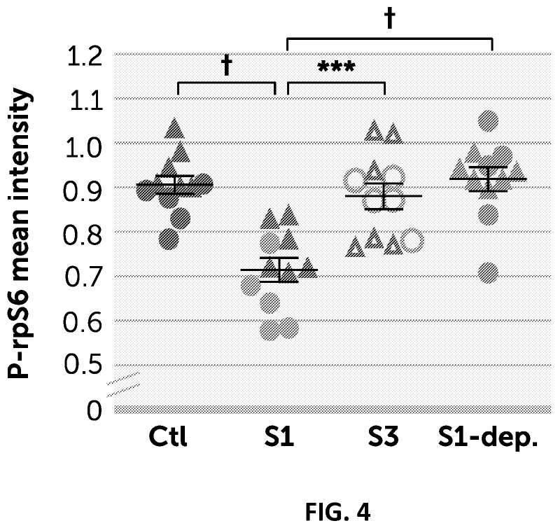

These convergent data provide robust evidence that children with PANDAS, as a group, possess antibodies that bind to CINs. To investigate these phenomena further, slices of mouse brain were kept alive in oxygenated artificial cerebrospinal fluid at 37° C. and treated for 1 hr with PANDAS or control serum; they were then fixed, sliced, and immunostained for phospho-rpS6, a marker of neural activity validated in CINs. Incubation with control serum had no effect on CIN activity (as indexed by P-rpS6), relative to saline (to which data were normalized). Treatment with baseline PANDAS serum reduced P-rpS6. This effect was lost in post-treatment serum from the same subjects, and in baseline serum from which IgG was depleted (FIG. 4). IgG binding to CINs correlated with reduced P-rpS6. There was no effect of serum on P-rpS6 in parvalbumin-expressing interneurons. Similar results were observed in a small number of sera characterized using an electrophysiological assay: preincubation of acute slices of mouse striatum reduced CIN response to bath-applied glutamate and serotonin, relative to preincubation with control serum. Thus, IgG in PANDAS serum can functionally inhibit CINs.

### Identification of Specific Target Antigens and Development of a More Quantitative Assay

These published data demonstrate that IgG from children with PANDAS show elevated binding to striatal CINs, that this binding declines in parallel with symptom improvement after treatment, and that IgG binding to CINs reduces their function. However, this immunofluorescence-based assay is only semi-quantitative, has high background, does not cleanly separate PANDAS from control samples, is laborious, and would be difficult to standardize.

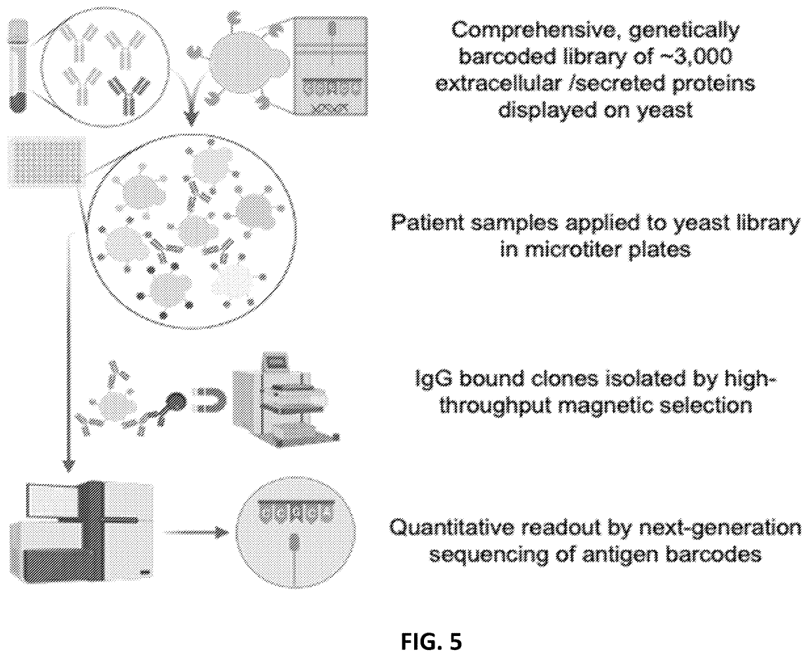

A modern screening technology, Rapid Exoproteome Antibody Profiling or REAP (FIG. 5), was utilized to determine patient antibody reactivities against thousands of human extracellular proteins simultaneously, in a single reaction. It adapts a concept from directed evolution, yeast surface display. A library of >3,000 extracellular and secreted human proteins was created, each represented in dozens of genetically barcoded yeast clones that present thousands of copies of a single human extracellular protein tethered to their surface. A small sample of patient serum (1-10 μg IgG) is applied to the library and magnetic selection is used to rapidly isolate yeast clones that are coated with IgG. The identities of IgG-bound proteins are then detected using next-generation sequencing (NGS) of the corresponding barcodes.

In unpublished work, REAP was applied to 11 subjects with PANDAS (cohorts 1 and 2 from Xu, J. et al. Antibodies From Children With PANDAS Bind Specifically to Striatal Cholinergic Interneurons and Alter Their Activity. Amer. J. Psych. 178, 48-64, doi:10.1176/appi.ajp.2020.19070698 (2021)), corresponding to red and orange symbols in FIGS. 1A-1B & 3A-3B). This preliminary screen identified high-affinity antibody binding to three candidate target antigens: LRP11 (found in 5 of 11 PANDAS samples and 0 controls), CXCL3 (2 of 11 PANDAS samples, 0 controls), and CSPG5 (1 PANDAS, 0 controls). These are not among the proteins previously proposed as antibody targets in PANDAS.

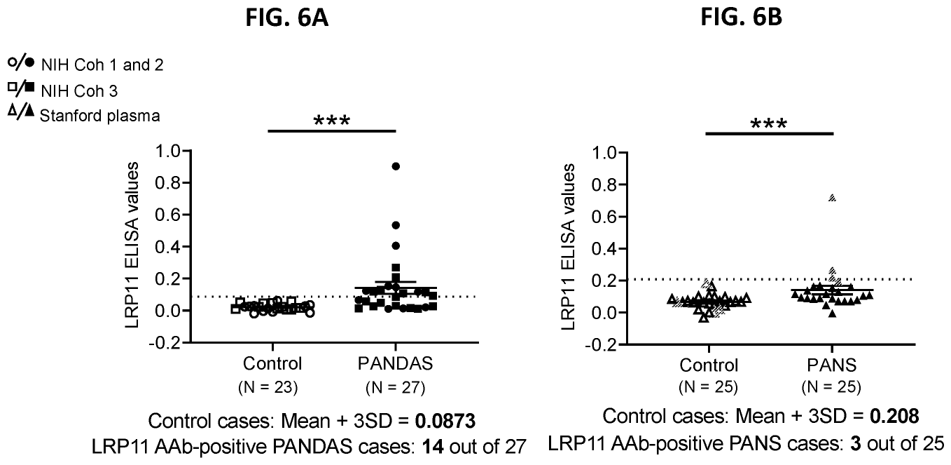

LRP11 was further characterized as a potential autoantibody target. Recombinant human LRP11 was purchased from a commercial source and used to develop and validate a fully quantitative enzyme-linked immunoassay (ELISA). Anti-LRP11 antibodies were significantly elevated in 50 NIMH PANDAS cases (FIG. 6A; this includes the 27 characterized in FIGS. 1A-1C); ELISA values fell outside a standard cutoff of control mean+3 SD in 27 of 50 PANDAS cases (14 of 27 from FIGS. 1A-1C) but in no controls. Anti-LRP11 antibodies were also elevated in the Stanford cohort, falling outside the cutoff (which was higher than in the NIMH samples due to elevated binding in 2 controls) in 3 of 25 cases and no controls (FIG. 6B). Anti-LRP11 antibodies are reduced after IVIG treatment in the NIMH cohort (p=0.032) and at remission in the Stanford cohort (p=0.01).

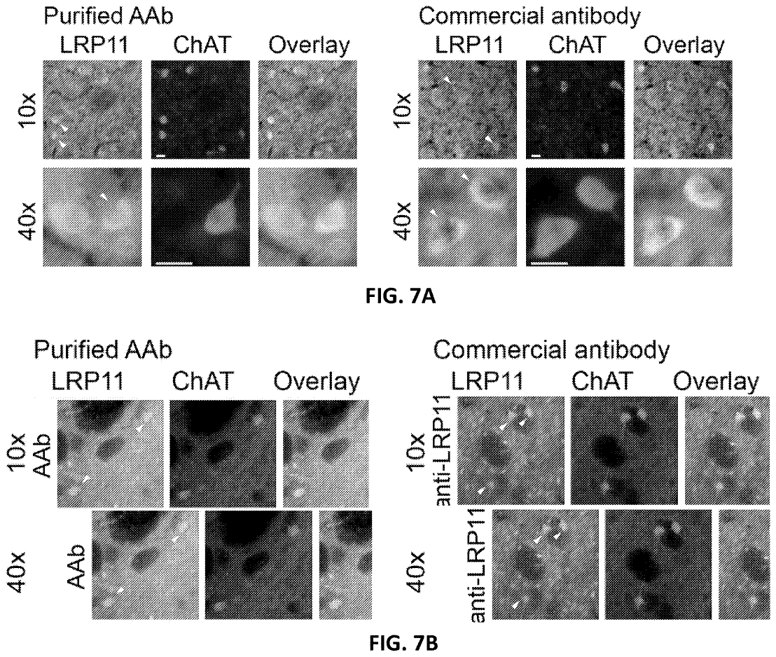

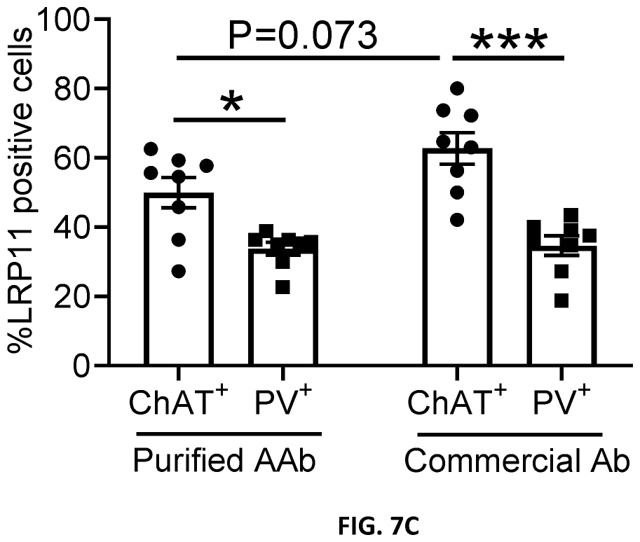

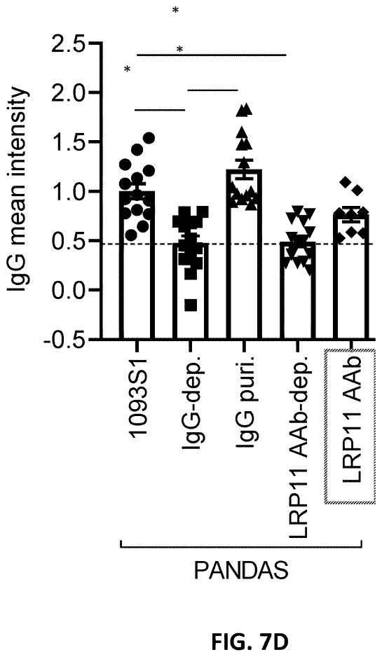

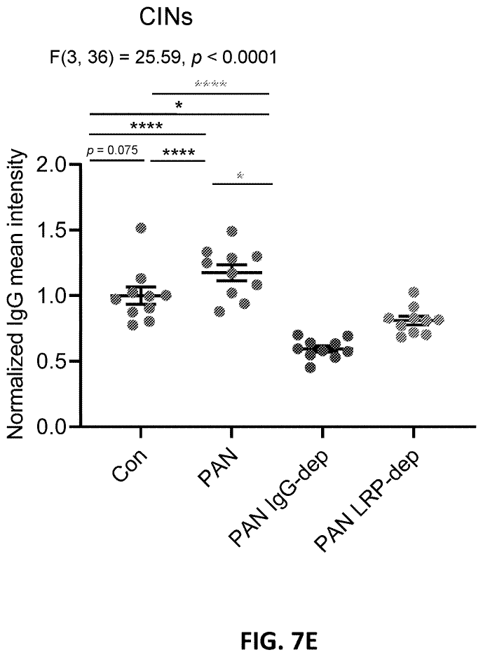

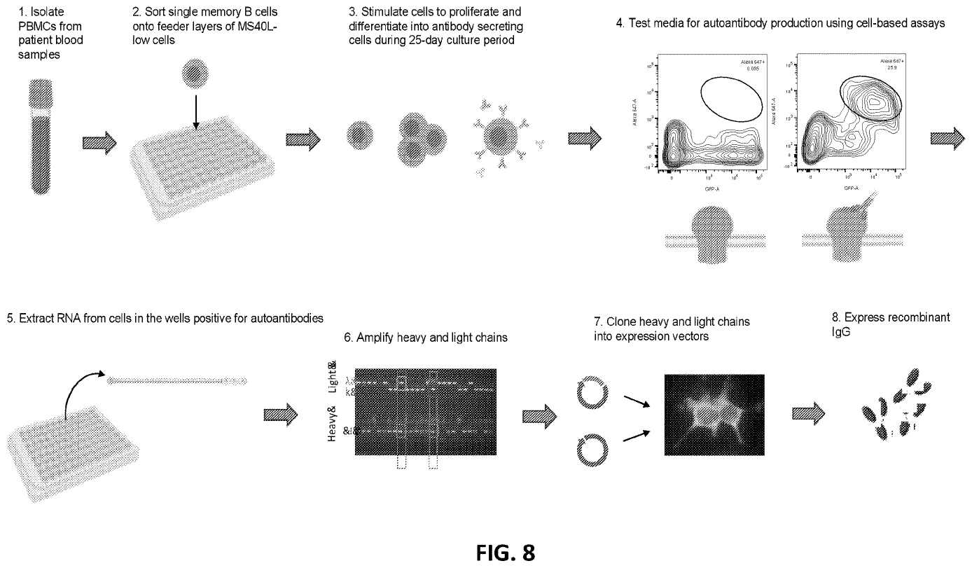

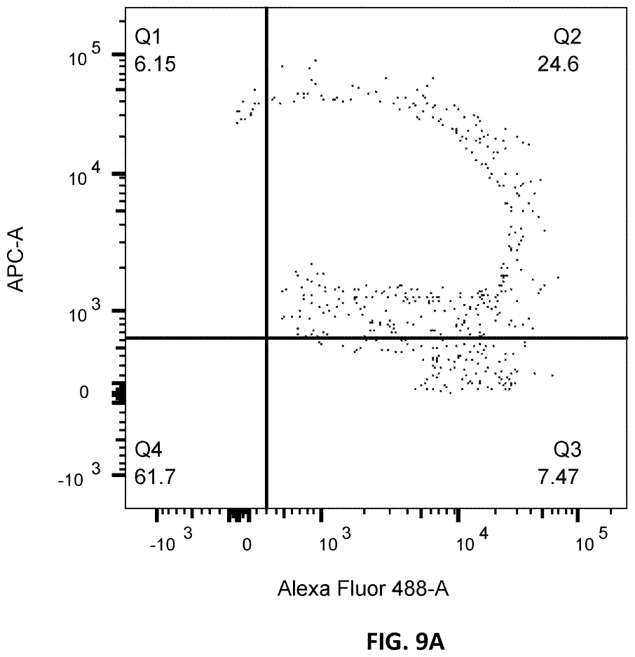

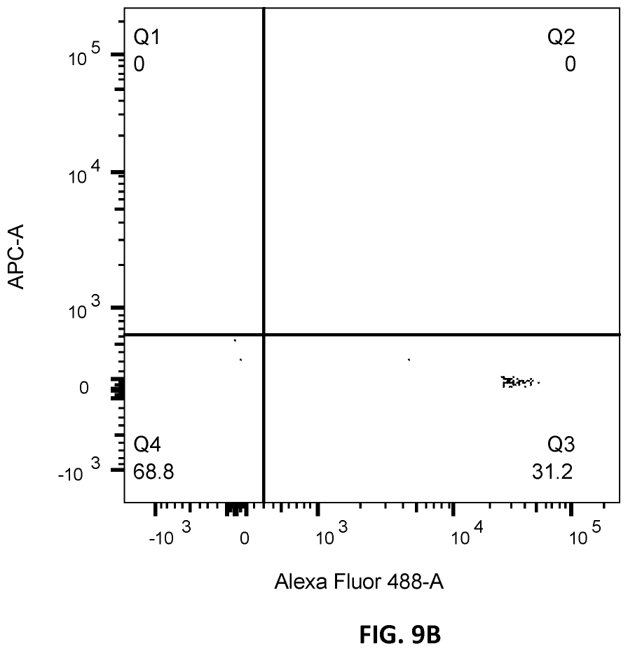

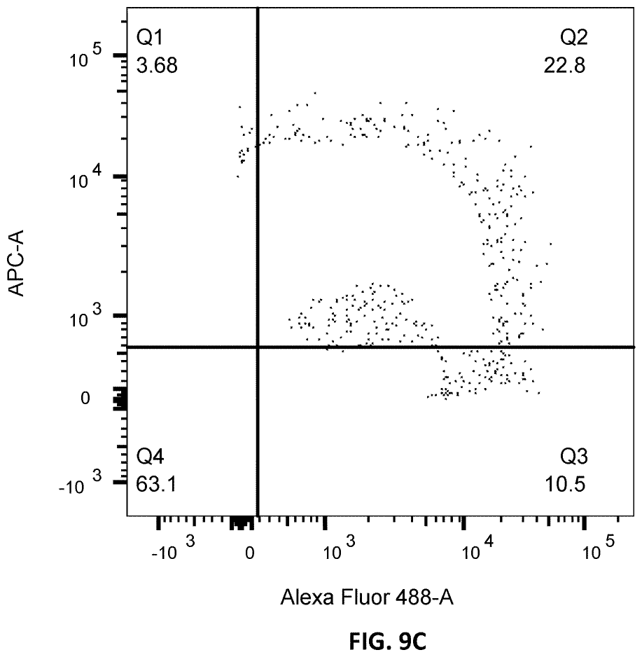

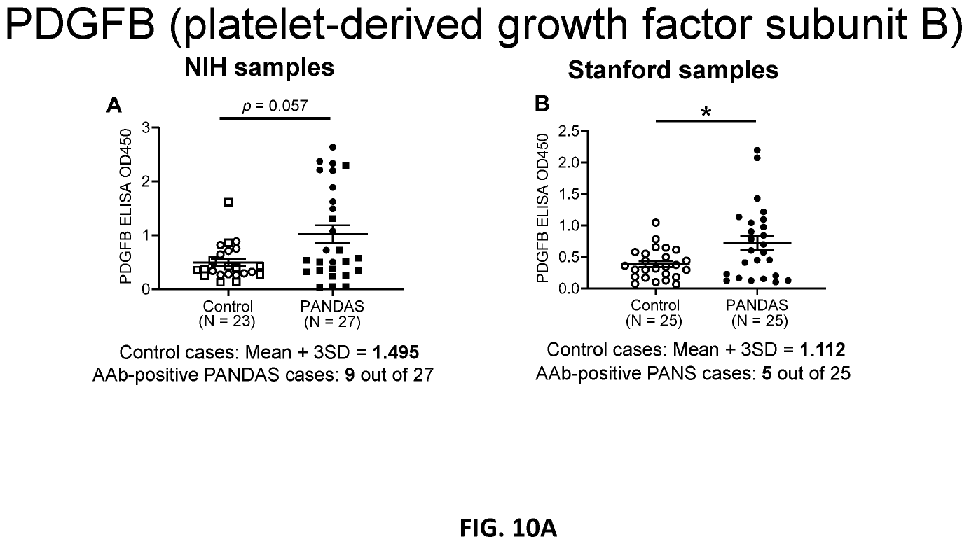

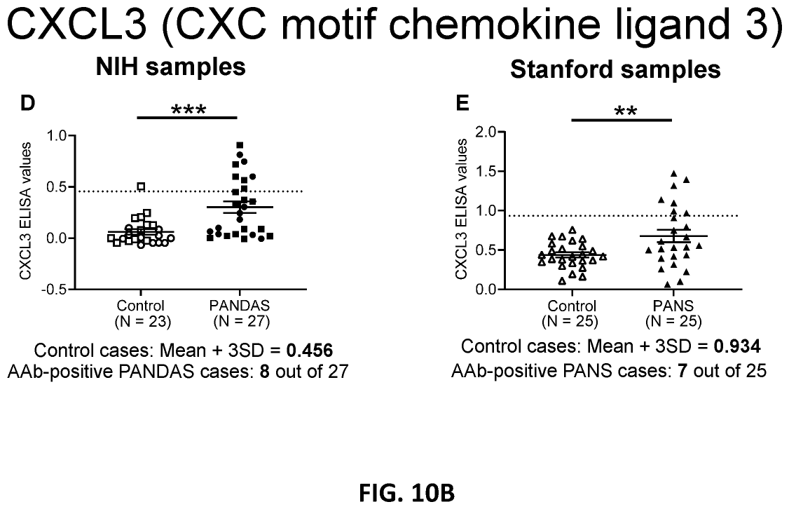

LRP11, or low-density lipoprotein receptor-related protein 11, is a transmembrane protein implicated in beta-catenin signaling (accession no. Q86VZ4). It has been little studied in the brain, but it is expressed in numerous brain regions, including in large striatal cells that are presumptive CINs (Allen Brain Atlas: 69837962; the CINs are by far the largest cells in the striatum and are readily recognizable based on their size and morphology). To test the contribution of anti-LRP11 antibody IgG binding to CINs in the immunofluorescence assay, we selected a high-LRP PANDAS serum (the highest binding serum in FIG. 6A) and depleted it of both all IgG (as in FIG. 4) and anti-LRP11 specific IgG (using agarose beads conjugated to recombinant human LRP11). Both total IgG and anti-LRP11 were also purified. Both commercial and PANDAS-derived anti-LRP11 antibodies bound to CINs, in both mouse (FIG. 7A) and human brain tissue (FIG. 7B). Binding was also seen to PV-interneurons, but the fraction of CINs positive for antibody binding was significantly higher (FIG. 7C). Depletion of either all IgG or just anti-LRP11 IgG significantly attenuated CIN binding (FIG. 7D). Incubation with purified total IgG or, to a lesser extent, with purified anti-LRP11 from serum recapitulated CIN binding (FIG. 7D). (Antibody titer in the purified anti-LRP11 condition is much lower than in the other conditions; quantitative comparison of this condition with the others is therefore difficult.)

Identifying and validating candidate targets allows the development of quantitative ELISA assays (FIGS. 6A-6B), which allows one to characterize large numbers of sera both more efficiently and much more quantitatively than the original semi-quantitative immunofluorescence-based approach. CIN binding (FIGS. 7A-7D) can serve as a filter to select the most likely candidates.

### Pandas and Pans

While the diagnosis of PANDAS presumes an association with Streptococcal infection, PANS, a newer diagnostic entity, makes no such assumptions; it simply describes the rapid onset of pediatric OCD (or anorexia), together with any of a range of accompanying symptoms that are often seen clinically in these children. (See elsewhere herein for full diagnostic criteria of both PANDAS and PANS.) Indeed, one of the motivations for the development of the PANS diagnosis was to step away from presumptions about pathophysiology. It is likely that some PANS cases that do not qualify for a diagnosis of PANDAS are associated with other infections, such as mycoplasma, Lyme, or influenza. Others may be associated with Streptococcus infections that have escaped detection; still others may have no infectious cause. A critical next step in the research program is the examination of anti-interneuron IgG in PANS cases that do not meet criteria for PANDAS.

### Pathophysiological Model of Pandas and Pans

Striatal CINs have been implicated in the development of disorders of repetitive behavior in two previous independent lines of work. Post-mortem analysis has revealed a ~50% reduction in CINs, and of certain other interneurons, in the caudate and putamen of individuals with Tourette syndrome. CINs were experimentally depleted in the dorsal striatum of mice (equivalent to the human caudate and putamen) to test the causal effects of this deficiency; CIN depletion in an otherwise normal adult mouse was found to produce repetitive behavioral pathology. Thus, CIN deficiency is independently associated with a related human disease, and it is causally sufficient to produce repetitive behavioral pathology.

In combination, these results indicate a specific, unexpected, and surprising pathophysiological model for PANDAS, and perhaps PANS more generally. Without being bound by theory, it is believed that individuals who develop PANDAS and PANS develop antibodies that bind to CINs (FIGS. 1A-1C and FIG. 2) and inhibit them (FIG. 4).

This disclosure contains several non-limiting conceptual and technical innovations, which individually or in combination position one to critically advance the understanding of PANS and PANDAS, with implications for mechanisms of neuroimmune pathophysiology in neuropsychiatric disease or disorder more broadly.

The finding of elevated IgG binding to striatal CINs in PANDAS (FIGS. 1A-1C and 2) represents a surprising and unexpected insight into the pathophysiology of this condition, moving the field forward after two decades of controversy. This finding was replicated in 4 cohorts of patients from 2 clinics on opposite sides of the country, in 3 different experimenters'hands, using 2 technical approaches, with strict blinding and both manual and automated quantification, on both mouse and human tissue. This rigor and replication justify confidence in the finding.

The initial focus on antibody binding to specific cells, rather than to specific molecules, is a new approach in this field. It provides a powerful tool to functionally analyze candidate molecular targets as they emerge.

By examining whether antibodies against candidate target antigens recapitulate the characteristics we have demonstrated in PANDAS serum-preferential binding to and inhibition of CINs-one can functionally screen these candidates, selecting those most likely to contribute to the pathophysiological model described above. A first instance of this approach is shown above (FIGS. 7A-7D). This use of a functional assay to screen potential targets represents a fundamental advance in the study of PANDAS.

### EXAMPLES

Various embodiments of the present application can be better understood by reference to the following Examples which are offered by way of illustration. The scope of the present application is not limited to the Examples given herein.

PANDAS is defined by (i) sudden onset or worsening of OCD symptoms and/or tics after Streptococcal infection (we require CY-BOCS≥16); (ii) prepubertal onset; (iii) episodic course; (iv) adventitious movements, such as physical hyperactivity or choreiform movements, during exacerbations. Additional symptoms, such as separation anxiety, bedwetting, handwriting deterioration, attentional symptoms, and trouble sleeping are common but are not part of the diagnostic criteria. PANS is defined by (i) abrupt onset of OCD symptoms and/or severely restricted food intake (here focusing in certain non-limiting embodiments on OCD symptoms, with CY-BOCS 24 16); (ii) concurrent presence of at least 2 categories of associated symptoms during exacerbations, including anxiety, mood lability, irritability or aggression, developmental regression, sudden deterioration in school performance, motor or sensory abnormalities, and sleep disturbance or urinary symptoms. There is no age restriction on the PANS diagnosis; herein we focus, in certain non-limiting embodiments, on children aged 4-18.

### Specimen Collection

These investigations of human plasma samples were approved by the Human Investigations Committees of Yale and Stanford Universities. Plasma was collected at the Stanford Immune Behavioral Health Clinic and Research Program, from local patients and controls (living in the 7 counties surrounding Stanford University). Patients were classified as meeting PANS criteria, with a relapsing and remitting course, by a child psychiatrist (MT or MS). Patients were selected who had a positive test for Streptococcus, though many did not meet full diagnostic criteria for PANDAS. Parents gave written informed consent to participate in the study, and competent subjects gave assent prior to blood collection. Serum samples were collected during symptom flare; in a subset of subjects, samples were also collected during a period of symptom recovery/remission, either before or after the flare (time between symptom onset and remission/recovery draws: 4.15±0.85 years). All samples were aliquoted prior to storage at the Stanford Biobank, re-aliquoted into smaller volumes upon arrival, and stored at −80° C. until use. All samples were anonymized before being used for analysis; all analyses were performed blind to diagnosis and condition. Samples/disease status classification was based on the data collected by the Clinician Encounter Form which clinicians/research staff complete at the end of the clinic visit after reviewing patient/parent questionnaires, psychometrics, and visit notes.

### Mouse and Human Brain Tissues

All experimental procedures were approved by the Yale University Institutional Animal Care and Use Committee, in accordance with the NIH Guide for the Care and Use of Laboratory Animals. Male and female C57BL/6J mice (adults 3-6 months old and juveniles 4-5 weeks old) were purchased from the Jackson Laboratory (Bar Harbor, Maine, http://jaxmice.jax.org/strain/013636.html). Mice were anesthetized by intraperitoneal injection of ketamine (100 mg/kg) and xylazine (10 mg/kg) and transcardially perfused with cold 4% paraformaldehyde in 1×PBS (pH 7.4). Brains were fixed overnight in 4% PFA at 4° C., followed by equilibration in 30% sucrose for 48 h at 4° C. Striatal slices were cut at 20 μm using a Leica CM3050S cryostat (Leica, Buffalo Grove, IL). Slices were stored in a cryoprotectant solution (30% glycerin, 30% ethylene glycol in 1×PBS pH 7.4) at −20° C. until use. Slices of human basal ganglia were collected and prepared as part of an unrelated study, as described previously.

### Reagents and Antibodies

Ketamine (Ketaset) was obtained from Zoetis (Madison, NJ). Xylazine (Anased) was obtained from Akorn, Inc. (Decatur, IL). Reagents for immunohistochemical staining (FIG. 1-3) were obtained from Sigma (St. Louis, MO: Sudan Black B, Triton X-100, sodium fluoride) or from JT Baker (Phillipsburg, NJ: paraformaldehyde). Normal donkey serum in blocking buffer was obtained from Jackson Immunoresearch (West Grove, PA). Chemicals used for acute brain slice analysis (FIG. 4) were obtained from Sigma (N-methyl-D-glucamine (NMDG), glucose, thiourea, sodium pyruvate, sodium ascorbate and HEPES) or JT Baker (KCl, NaCl, NaH₂PO₄, NaHCO₃, MgSO₄ and CaCl₂).

### Determination of IgG Titers in Plasma Samples

Total IgG titers in plasma samples were determined using IgG (Total) Human ELISA Kit (Thermo Scientific, Rockland, IL) following manufacturer's instructions and as described [46]. More details are described in the Supplementary Information. Total IgG titers were higher in control samples than in PANS samples; there were no differences between genders or between PANS “flare” and “recovery” samples. All plasmas were diluted in 1×PBS+0.1% BSA (bovine serum albumin) to 500 mg/dL IgG and randomized before testing. An equal amount of diluted plasma, and thus of IgG, was used in all assays.

### Assessing Phospho-rps6 Levels in Acute Mouse Brain Slices

Acute coronal mouse brain slices containing the striatum were prepared from male and female C57BL/6J mice as previously described. Briefly, mouse brains were quickly removed and placed in ice-cold oxygenated NMDG-aCSF (artificial cerebrospinal fluid, in mM: 92 NMDG, 2.5 KCl, 1.25 NaH₂PO₄, 30 NaHCO₃, 20 HEPES, 10 MgSO₄, 0.5 CaCl₂, 25 glucose, 2 thiourea, 3 sodium pyruvate and 5 sodium ascorbate, pH 7.35, saturated with 95% O₂/5% CO2). Coronal slices (100 μm) through the striatum were cut using a Leica VT1000S vibratome (Leica Microsystems, Bannockburn, IL, USA) in the NMDG-aCSF solution. Slices were recovered in NMDG-aCSF for 10 min at 32° C. before being transferred to regular aCSF (in mM: 119 NaCl, 2.5 KCl, 1.25 NaH₂PO₄, 24 NaHCO₃, 2 CaCl₂, 2 MgSO₄ and 12.5 glucose) for 1 h at 30° C. under constant oxygenation with 95% O2/5% CO2. After recovery, slices were treated with plasma samples (6.25 mg/dL diluted in aCSF) or the aCSF control for 1 h at 30° C. After treatment, slices were fixed in cold 4% paraformaldehyde (PFA) in 1×PBS (pH 7.4) containing 5 mM NaF for 1 h at 4° C., followed by immunohistochemistry.

### Immunohistochemistry and Image Quantification

Paraformaldehyde-fixed mouse striatal slices (20 μm coronal sections) and formalin-fixed human brain slices (50 μm coronal sections of caudate and putamen) were used to examine plasma IgG deposition, as described. Brain sections were blocked in freshly prepared 0.1% Sudan Black B (in 70% ethanol) for 10 min at RT to reduce autofluorescence. After washes, sections were incubated in blocking buffer (1×PBS+0.3% Triton X-100 supplemented with 5% normal donkey serum) for 1h at RT, and then incubated with plasma (1.25 mg/dL each in blocking buffer) overnight at 4° C. The next day, sections were washed in blocking buffer and incubated with an anti-human IgG antibody and specific neuronal markers (ChAT and PV) overnight at 4° C., followed by incubation with fluorophore-conjugated secondary antibodies for 1 h at RT. After washes, sections were mounted in Vectashield HardSet Mounting Medium (Vector Laboratories), coverslipped, and stored at 4° C.

For plasma IgG binding to mouse neurons (FIG. 2), each plasma was tested in brain sections from 6 mice (both male and female), with 4-6 images collected randomly from the dorsal striatum of each mouse, without overlap, using an Axio Scope A1 fluorescent microscope with a 10×/0.45 NA objective (Zeiss, Germany). For plasma IgG binding to human brain slices, each plasma was tested in brain sections from 2 subjects, with 6 images collected, without overlap, in each section. Images were captured by sequential scanning of sections on an Olympus Fluoview FV-1000 confocal microscope with a 20×/0.85NA objective (Olympus, Japan).

### Identification and Functional Testing of Candidate Molecular Targets

The elevated binding of PANDAS serum IgG to CINs was discovered by analyzing binding to specific cell types, rather than candidate molecules. Now that a specific cellular target, the CINs, was identified and multiply replicated the critical, next step is the identification of the molecular target, or targets, to explain this effect.

The field has been searching for the causative antibodies in PANDAS (and more recently in PANS) for twenty years. A number of candidates have been proposed, largely by analogy to Sydenham chorea-D1 and D2 dopamine receptors; tubulin; lysoganglioside G_{M1}. Some studies have found antibodies against these targets in PANDAS patients. However, these targets have not been universally replicated. For example, elevated anti-D2R receptor antibodies have been reported in three studies but not in three others. The present study does any elevated binding against striatal medium spiny neurons expressing D1 or D2 dopamine receptors. An unbiased approach for the identification of antibody targets in PANDAS and PANS is needed.

### The REAP Screening Approach

An approach to screening for pathogenic antibody targets should ideally have certain characteristics. In one aspect, it should be unbiased. In one aspect, it should screen for binding to proteins in their native conformation, rather than in denatured form; testing for binding of antibodies in denaturing gels, for example, is likely to miss many potential targets. In one aspect, it should allow for the detection of targets that are present at very low levels; CINs represent only ~1% of the neurons in the striatum, and thus important proteins on them may represent a tiny fraction of all protein in striatal lysates.

Rapid Exoproteome Antibody Profiling, or REAP, uses yeast surface display to test for antibody binding to thousands of human extracellular and secreted proteins, representing nearly the entire human exoproteome, in a rapid, unbiased, massively parallel screen. This approach was applied to the first 11 PANDAS samples, and 5 matched controls, and three candidate targets identified: LRP11 (found in 5 of 11 PANDAS samples and 0 controls), CXCL3 (2 of 11 PANDAS samples, 0 controls), and CSPG5 (1 PANDAS, 0 controls). These are the first three candidates to be tested; initial characterization of anti-LRP11 is presented elsewhere herein (FIGS. 6A-6B, 7A-7D). Of note, antibodies that bind to LRP11 have not been detected in >600 sera from both healthy subjects and patients with a range of other diagnoses in studies performed using the REAP assay to date. In parallel, antibodies against previously proposed targets are tested: D1 and D2 dopamine receptors, tubulin, and lysoganglioside G_{M1}. REAP is performed on all >350 sera being screened (see elsewhere herein, Table 1) to identify additional candidate pathogenic targets.

For each of the >3000 screened target proteins, for each serum, the REAP process assigns a binding affinity score from 0 (no binding detected) to 5 (strong binding). To select targets from this large dataset for further characterization the following principles are used. In certain non-limiting embodiments, targets with strong binding (4 or 5) in one or more PANDAS, PANS, or OCD samples, and not in control samples, are prioritized. In certain non-limiting embodiments, targets found in multiple clinical samples are prioritized. In certain non-limiting embodiments, targets for which binding is also seen in control samples are deprioritized, although these may still be investigated if they are found in a substantially higher percentage of clinical than control sera. Following these principles, up to 40 potential molecular targets in PANDAS and/or PANS will be identified.

### Validation of REAP Hits

For each target an ELISA assay (for secreted proteins) or flow-based cellular assay (for selected membranous proteins) are developed, allowing rapid quantitative measurement of antibody binding. Anti-LRP11 in these sera has been confirmed by ELISA (FIGS. 6A-6B). One can raise or acquire polyclonal antibodies for the contemplated testing.

### Interneuron Binding Assay

Polyclonal antibodies are raised against candidate targets identified by REAP, or purchased from commercial sources, where available. One can test the ability of these polyclonal antibodies to bind to CINs in the ex vivo binding assay (FIGS. 1A-1C, 2, and 7A-7D), both with antibody alone, across 4 logs of concentration, and with antibody in healthy control serum. Pre-immune serum and polyclonal antibodies to proteins not identified as targets by REAP serve as negative controls. If binding is seen in the presence of control serum but not when antibody is added alone, this may suggest in certain embodiments that other components of serum (e.g. crosslinking antibodies) are required for binding to manifest.

Briefly, 20 μm floating coronal cryostat sections through the dorsal striatum of PFA-perfused wild-type mouse brains are post-fixed, rinsed, quenched (to remove autofluorescence), blocked, and then incubated overnight at 4° C. with antibody across several orders of magnitude of concentration or with antibody+serum (total serum IgG concentration of 1.25 mg/dL) in a standard immunohistochemistry buffer (saline with added detergent and 5% donkey serum to block nonspecific binding by secondary antibody). Slices are then rinsed and incubated for 1 hr at room temperature with fluorophore-conjugated secondary antibodies, rinsed again, and mounted on slides. Binding is visualized using confocal imaging and quantified as in the published work and detailed elsewhere herein.

### CIN Activity Assay

Antibodies that bind to CINs are further characterized for their ability to inhibit CIN activity in vitro, as in FIG. 4. Briefly, 100 μm coronal slices through the striatum are produced from wild-type mice and allowed to recover at 30-32° in artificial cerebrospinal fluid. They are then treated with antibody, with or without serum at 6.25 mg/dL (a concentration we arrived at empirically in pilot experiments) for 60 min at 30°, or with vehicle with no serum, before being fixed in paraformaldehyde with phosphatase inhibitors. Slices are immunostained for P-rpS6 and interneuronal markers (ChAT or PV); P-rpS6 levels are quantified following the same procedure we use to quantify IgG binding, detailed below. This methodology is again optimized to ensure rigor. All staining is quantified blind to experimental condition. Each serum is tested in 4-6 slices, which are averaged to produce an aggregate value. Tests are run in batches balanced between control and clinical samples, with saline control slices in each; P-rpS6 levels are normalized to the mean value from saline-treated slices run in parallel, eliminating any batch effects.

### Electrophysiological Effects of Antibodies Against Identified Targets

PANDAS serum was found to alter the responses of CINs, using in vitro patch clamp electrophysiology. This analysis was severely limited because it requires the use of large amounts of serum (100-200 μL). The identification of candidate molecular targets, and the development of polyclonal antibodies against them, allows one to overcome this impediment. Acute slices of mouse dorsal striatum are prepared, pretreated with normal serum with or without antibody against selected targets (across a 2-log concentration range), and CIN electrophysiological properties are measured at baseline and after application of AMPA, dopamine, and serotonin. Based on the results from a single serum pair (one PANDAS, one control), in certain non-limiting embodiments, pretreatment with antibodies that bind to CINs and reduce P-rpS6 can also reduce CINs'response to bath-applied AMPA. Elucidating the specific electrophysiological effects of antibody binding helps refine the understanding of the effects of PANDAS-related (and perhaps PANS-related) antibodies on striatal microcircuitry and information processing.

### Analysis and Power Considerations

REAP produces semi-quantitative measures of antibody binding to >3,000 targets, representing most of the human exoproteosome. Candidate targets are selected as described elsewhere herein. Once polyclonal antibodies have been raised against these candidates, they are compared to pre-immune serum and to polyclonal antibodies raised against control antigens (REAP negative) in CIN binding and P-rpS6 assays. In both cases, one can vary antibody concentrations across four orders of magnitude. The antibody concentration that produces the maximum signal to noise in CIN binding (relative to background fluorescence) is used as the starting concentration for P-rpS6 and electrophysiological assays; concentration is varied systematically in these assays as well. Key analyses are between-group comparisons of selected antibodies vs off-target IgG control; these are performed by independent samples, 2-tailed t-test, or by Mann-Whitney U test if distributions are non-normal. If multiple targets are identified and validated, their characteristics (e.g. specificity to CINs, ability to reduce CIN activity, qualitative and quantitative effects on CIN electrophysiological properties and responses) are examined in relation to clinical variables, as described.

### Alternate Strategies

Alternative methods for the identification of antibody targets are available. For example, in certain non-limiting embodiments, REAP may not be optimal for detection of antigens characterized by extensive post-translational modification that is not fully recapitulated in the yeast surface display system. As an alternative, should one not get adequate hits using REAP, one can enrich CINs and then test for antibody binding to CIN proteins using standard proteomic methods. CINs can be enriched using FACS sorting of triturated striatal tissue, or by differentiating them from human IPS cell.

### Characterization of Antibody Binding to CINs Across PANS, PANDAS, and OCD

IgG from patients with PANDAS, collected at two different clinical sites, binds to CINs at higher levels than IgG from controls (FIGS. 1A-1C and FIG. 2), and that this binding inhibits their activity (FIG. 4). One can investigate clinical measures (age, sex, racial background, serological and inflammatory measures, symptomatology) as correlates of IgG binding. In certain non-limiting embodiments, this can further refine hypotheses as to the clinical effects of CIN binding and help one move towards mechanistically-based diagnostic clarification.

Interneuron binding assay. IgG binding to interneurons is quantified (FIGS. 1A-1C) and as detailed elsewhere herein. Sera are diluted to a uniform IgG concentration of 1.25 mg/dL. Following serum incubation, slices are incubated with anti-human IgG and anti-ChAT (or anti-PV) primary antibodies, followed by fluorescent-conjugated secondary antibodies.

Slices can be visualized using either confocal or standard fluorescence imaging. Confocal imaging were used in the original pilot study of five subjects, but standard fluorescence imaging has been used for the ongoing work; it has higher background but allows for much higher throughput (both more sera, and an order of magnitude more cells counted for each serum). A completely automated strategy has been developed for quantification of IgG binding to defined interneuronal populations; briefly, ChAT (or PV) immunoreactivity is thresholded and used to define ROIs corresponding to cell bodies of the corresponding neuron type, and IgG immunoreactivity is quantified within these individual ROIs, after background subtraction. For analysis, all cells within each slice are averaged, and then all slices incubated with each serum are averaged; the N for statistical comparisons is thus the number of sera, not the number of slices or cells quantified.

Several aspects of this methodology ensure rigor. Even though the quantification is automated, all assays are performed blind to diagnosis and experimental condition, providing an additional safeguard against bias. All sera are tested on slices from 4-6 mice; 36-48 non-overlapping microscope images are quantified for each serum. The total number of ChAT-positive cells and total ChAT immunoreactivity are measured, to ensure that there is no difference between groups in the number of ChAT cells present in the tissue. Sera are tested in batches of 10; each batch is balanced amongst clinical groups and is processed as a unit (with identical reagents, ambient temperature and other conditions, timing, and so forth). All samples analyzed in a particular batch of 10 are normalized to mean values from slices treated with control serum within that batch, eliminating batch effects.

### ELISA Assays

Better signal to noise is achieved by ELISA assays against selected targets, starting with anti-LRP11 (FIGS. 6A-6B).

### Cin Activity Assay

Binding by PANDAS-associated IgG is associated with reduced activity in CINs, as quantified using immunostaining to P-rpS6 (FIG. 4). The activity assay is lower-throughput than the IgG binding assay; it takes much longer per serum sample. The activity assay will not thus be run on every serum sample; rather, samples with high levels of CIN binding to test (~25% of the samples) will be selected, in comparison to selected control samples and a small number of clinical samples showing low CIN binding. If the clinical groups that are being tested for the first time here (PANS; OCD) do not show elevated IgG binding to CINs, a random subset of sera will be tested, matched to those selected from the PANDAS group.

### Analysis and Power Considerations

As detailed above, measures of IgG binding to interneurons and of the effect of serum pretreatment on interneuronal P-rpS6 are derived from multiple cells in multiple slices, normalized to within-batch control conditions. The unit of analysis is the serum, rather than the cell, slice, or replicate, in all experiments. Primary analyses are between-group comparisons, which are performed by independent samples t-test (for 2 groups) or ANOVA (for mulitple groups), as in FIGS. 1A-1C, 2, and 4. Nonparametric alternatives are used if data are non-normal, which is determined using the Kolmogorov-Smirnov test. Across all samples one is very well powered; one has 80% power to detect effects in the PANS and PANDAS groups, relative to controls, of d≥0.39, and 95% power to detect d≥0.5 (these are for two-tailed tests, although one-tailed tests could easily be justified given the pilot data). Since the effect seen in the data to date is large (FIGS. 1A-1C: d=1.2), there is confidence that clinically relevant effects can be detected in these groups. Correlations between IgG binding and effects on CIN activity (i.e. P-rpS6 levels) are performed by Pearson correlation (or Spearman if data are non-normal). Exploratory analyses examine the relationship between IgG binding (or effect on P-rpS6) and clinical variables using linear and logistic regression models. Candidate predictors include severity of symptoms (CY-BOCS), duration of symptoms, age, age at symptom onset, sex, presence of other immunological abnormalities, present of neuropsychiatric comorbidities, evidence for infection with specific agents (Streptococcus, mycoplasma, Lyme, influenza, other, none/unknown). In correlational analyses one has 80% power to detect relationships of |ρ|≥0.175 (two-tailed), and 95% power to detect |ρ|≥0.223.

### Selection of Subjects for B Cell Screening and mAb Isolation

This process of B-cell isolation and mAb cloning is laborious and cannot be done at scale; it is therefore necessary to carefully select patient samples for screening. At least 20 patient samples are screened over the course of the 5-year funding period. Subjects with PANDAS or PANS are selected on the basis of (i) severe illness, (ii) strong binding to CINs, (iii) clear identification of a target, (iv) confirmation of antibody in PANDAS or PANS serum using ELISA (FIGS. 6A-6B) and (v) if possible, clear evidence that polyclonal antibodies against the target have CIN binding and CIN inhibition activity (FIGS. 7A-7D).

### Testing of Candidate Patient mAbs for Anti-CIN Activity

Well-established ex vivo assays for IgG binding to CINs (FIGS. 1A-1C and FIG. 2) and IgG inhibition of CIN activity (FIG. 4) are used to test the effects of cloned IgG autoantibodies identified here. Cloned IgG that binds to and inhibits CINs become the strongest candidate for a specific causative antibody in PANDAS and/or PANS.

### Enumerated Embodiments

The following enumerated embodiments are provided, the numbering of which is not to be construed as designating levels of importance:

Embodiment 1 provides a method for diagnosing a neuropsychiatric disease or disorder in a human subject, the method comprising:
i) obtaining a serum sample comprising IgG from the human subject;
ii) quantifying using ELISA the amount of an autoantibody binding to the target antigen set to provide an ELISA value, wherein the autoantibody comprises at least one of anti-LRP11 antibody, anti-CXCL3 antibody, an anti-PDGFB antibody, and anti-CSPG5 antibody;
iii) diagnosing the human subject with PANS if the ELISA value is greater than or equal to 3 standard deviations above a control mean ELISA value.

Embodiment 2 provides the method of embodiment 1, wherein the neuropsychiatric disease or disorder is acute-onset neuropsychiatric syndrome (PANS) or pediatric autoimmune disorder associated with Streptococcus (PANDAS).

Embodiment 3 provides the method of any one of embodiments 1-2, wherein the target antigen is LRP11.

Embodiment 4 provides the method of any one of embodiments 1-3, wherein the target antigen is CXCL3.

Embodiment 5 provides the method of any one of embodiments 1-4, wherein the target antigen is CSPG5.

Embodiment 6 provides the method of any one of embodiments 1-5, wherein the target antigen is PDGFB.

Embodiment 7 provides the method of any one of embodiments 1-6, wherein the autoantibody is an anti-LRP11 antibody.

Embodiment 8 provides the method of any one of embodiments 1-7, wherein the autoantibody is an anti-CXCL3 antibody.

Embodiment 9 provides the method of any one of embodiments 1-8, wherein the autoantibody is an anti-CSPG5 antibody.

Embodiment 10 provides the method of any one of embodiments 1-9, wherein the autoantibody is an anti-PDGFB antibody.

Embodiment 11 provides the method of any one of embodiments 1-10, further comprising treating the human subject to reduce or ameliorate at least one symptom of the neuropsychiatric disease or disorder.

Embodiment 12 provides the method of embodiment 11, wherein the treating comprises administering to the human subject a therapeutically effective amount of at least one of IVIG, rituximab, steroids, and non-steroidal anti-inflammatory drugs (NSAIDs).

Embodiment 13 comprises a method for identifying a disease-inducing agent in a neuropsychiatric disease or disorder, the method comprising:
i) providing an isolated tissue sample containing cholinergic interneurons (CINs);
ii) identifying one or more antibodies in a serum specimen that bind to the CINs;
iii) quantifying using ELISA the amount of one or more antibodies that bind to the CINs to provide an ELISA value;
iv) comparing the ELISA value against a control ELISA value to determine whether the one or more antibodies are disease-inducing agents.

Embodiment 14 provides the method of embodiment 13, wherein the isolated tissue sample is mammalian tissue.

Embodiment 15 provides the method of any one of embodiments 13-14, wherein the neuropsychiatric disease or disorder is PANS.

Embodiment 16 provides the method of any one of embodiments 13-15, wherein the neuropsychiatric disease or disorder is PANDAS.

Embodiment 17 provides the method of any one of embodiments 13-16, further comprising treating a human subject to reduce or ameliorate at least one symptom of the neuropsychiatric disease or disorder if the one or more antibodies are disease-inducing agents.

Embodiment 18 provides the method of embodiment 17, wherein the treating comprises administering to the human subject a therapeutically effective amount of at least one of IVIG, rituximab, a steroid, and a non-steroidal anti-inflammatory drug (NSAID).

What is claimed is:

1. A method for diagnosing a neuropsychiatric disease or disorder in a human subject, the method comprising:
i) obtaining a serum sample comprising IgG from the human subject;
ii) quantifying using ELISA an amount of an autoantibody binding to the target antigen set in the serum sample, so as to provide an ELISA value, wherein the autoantibody comprises at least one of an anti-LRP11 antibody, an anti-CXCL3 antibody, an anti-PDGFB antibody, and an anti-CSPG5 antibody; and
iii) diagnosing the human subject with PANS if the ELISA value is greater than or equal to 3 standard deviations above a control mean ELISA value.

2. The method of claim 1, wherein the neuropsychiatric disease or disorder is acute-onset neuropsychiatric syndrome (PANS) or pediatric autoimmune disorder associated with Streptococcus (PANDAS).

3. The method of claim 1, wherein the target antigen is LRP11.

4. The method of claim 1, wherein the target antigen is CXCL3.

5. The method of claim 1, wherein the target antigen is CSPG5.

6. The method of claim 1, wherein the target antigen is PDGFB.

7. The method of claim 1, wherein the autoantibody is an anti-LRP11 antibody.

8. The method of claim 1, wherein the autoantibody is an anti-CXCL3 antibody.

9. The method of claim 1, wherein the autoantibody is an anti-CSPG5 antibody.

10. The method of claim 1, wherein the autoantibody is an anti-PDGFB antibody.

11. The method of claim 1, further comprising treating the human subject to reduce or ameliorate at least one symptom of the neuropsychiatric disease or disorder.

12. The method of claim 12, wherein the treating comprises administering to the human subject a therapeutically effective amount of at least one of IVIG, rituximab, a steroid, and a non-steroidal anti-inflammatory drug (NSAID).

13. A method for identifying a disease-inducing agent in a neuropsychiatric disease or disorder, the method comprising:
i) providing an isolated tissue sample containing cholinergic interneurons (CINs);
ii) identifying one or more antibodies in a serum specimen that bind to the CINs:
iii) quantifying using ELISA an amount of the one or more antibodies that bind to the CINs so as to provide an ELISA value; and
iv) comparing the ELISA value against a control ELISA value to determine whether the one or more antibodies are disease-inducing agents.

14. The method of claim 13, wherein the isolated tissue sample is a mammalian tissue.

15. The method of claim 13, wherein the neuropsychiatric disease or disorder is PANS.

16. The method of claim 13, wherein the neuropsychiatric disease or disorder is PANDAS.

17. The method of claim 13, further comprising treating a human subject to reduce or ameliorate at least one symptom of the neuropsychiatric disease or disorder if the one or more antibodies are disease-inducing agents.

18. The method of claim 17, wherein the treating comprises administering to the human subject a therapeutically effective amount of at least one of IVIG, rituximab, a steroid, and a non-steroidal anti-inflammatory drug (NSAID).
